(19) United States
(12) Patent Application Publication
(10) Pub. No.: US 2009/0117176 A1
(43) Pub. Date: May 7, 2009

(54) Anticancer Treatments

(75) Inventors: Erard Gilles, Bridgewater, NJ (US); Lars-Axel Sternas, Verona, NJ (US); Ovid Trifan, Bridgewater, NJ (US); Helgi van de Velde, Beerse (BE); April Teitelbaum, Doylestown, PA (US)

Correspondence Address: KING & SPALDING, 1185 AVENUE OF THE AMERICAS, NEW YORK, NY, 10036-4003, US

(73) Assignee: Pharma Mar, S.A. Sociedad Unipersonal, Madrid (ES)

(21) Appl. No.: 11/577,790

(22) PCT Filed: Oct. 26, 2005
(86) PCT No.: PCT/GB05/50189

§ 371 Date: Sep. 24, 2007

Related U.S. Application Data
(60) Provisional application No. 60/622,163, filed on Oct. 26, 2004.

(51) Int. Cl. A61K 9/127; A61P 35/00; A61K 31/704; A61K 31/4995

(52) U.S. Cl. 424/450; 514/34; 514/250

(57) ABSTRACT

A method of treating the human body for cancer comprises administering an effective therapeutic amount of a Pegylated Liposomal form of the anthracycline Doxorubicin (“PLD”), in combination with an effective therapeutic amount of ET-743

The present invention relates to the treatment of cancers and, in particular, an effective treatment of human cancers using Ecteinascidin 743 (ET-743) in combination with another drug.

Ecteinascidin 743 (ET-743) is an anticancer agent derived from a marine source.

### BACKGROUND OF THE INVENTION

Ecteinascidin 743 (ET-743) is a tetrahydroisoquinoline alkaloid isolated from the marine tunicate Ecteinascidia turbinata with the following structure:

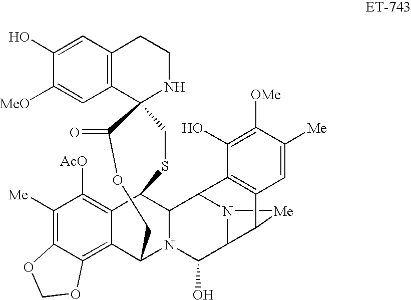

ET-743, its chemistry, mechanism of action and preclinical and clinical development can be found in Kesteren. Ch. Van et al. 2003, Anti-Cancer Drugs, 14 (7), pages 487-502: “Yondelis (trabectedin, ET-743): the development of an anticancer agent of marine origin”, and references therein.

ET-743 possesses potent antineoplastic activity against a variety of human tumour xenografts grown in athymic mice, including melanoma and ovarian and breast carcinoma.

In clinical phase I studies of ET-743, promising responses were observed in patients with sarcoma and breast and ovarian carcinoma. Therefore this new drug is currently under intense investigation in several phase II clinical trials in cancer patients with a variety of neoplastic diseases.

ET-743 has myelotoxic and hepatotoxic side effects. Patients who received ET-743 by prolonged infusion over 24-72 hr experienced myelosuppression and, frequently, acute, albeit reversible, elevation of transaminases and subclinical cholangitis characterized by increases in alkaline phosphatase (ALP) and/or bilirubin, see for example Ryan D. P. et al., 2001 Clin Cancer Res 7, 231: “Phase I and pharmacokinetic study of ecteinascidin 743 administered as a 72-hour continuous intravenous infusion in patients with solid malignancies”; Puchalski T. A. et al., 2002, Cancer Chemother Pharmacol 50: 309: “pharmacokinetics of ecteinascidin 743 administered as a 24-h continuous intravenous infusion to adult patients with soft tissue sarcomas: associations with clinical characteristics, pathophysiological variables and toxicity”.

Preclinical acute toxicity studies conducted in mice, rats, dogs and monkeys consistently demonstrated liver toxicity as an important side effect of ET-743, as evidenced by an increase in plasma levels of liver-specific enzymes and pathological manifestations of cholangitis. Recently the nature and extent of the hepatobiliary changes exerted by ET-743 in the female rat, the species which is most susceptible towards the hepatotoxic potential of ET-743, has been characterized by histopathology, electron microscopy, immunohistochemistry, plasma biochemistry and DNA microarray analysis, see Donald S. et al., 2002, Cancer Research, 62: 4256 “Hepatobiliary damage and changes in hepatic gene expression caused by the antitumor drug ecteinascidin 743 (ET-743) in the female rat”.

Furthermore, pretreatment with high-dose dexamethasone has been shown to abrogate ET-743-mediated hepatotoxicity in this animal model without impeding its antitumor activity, see Donald S. et al., 2003, Cancer Research, 63: 5903-5908: “Complete protection by high-dose dexamethasone against the hepatotoxicity of the novel antitumor drug ecteinascidin-743 (ET 743) in the rat” and WO 02 36135. Protection by dexamethasone pretreatment was accompanied by a dramatic reduction in hepatic levels of ET-743, tentatively implicating elevated hepatic clearance of ET-743, perhaps via induced metabolic enzymes, as the mechanism by which dexamethasone exerts its beneficial effect, ie via an increase in the rate of metabolic detoxification of ET-743.

Doxorubicin is a cytotoxic anthracycline antibiotic isolated from Streptomyces peucetius van caesius. Doxorubicin is known to cause primarily myelotoxicity when administered alone.

The reader is referred to WO 02 36135, published 10 May 2002 and incorporated herein by specific reference, for compositions and uses of ET-743 with other drugs for treating cancer. In vitro assays indicated more than additive effects for the combination of ET-743 with several other drugs. In particular, synergistic effects were shown in vitro against human sarcoma tumours. Compositions and uses of ET-743 with the anthracycline Doxorubicin were investigated. In this study there was no detailed consideration of the toxicity of combinations.

Further guidance on the dosage, schedules and administration of ET-743 alone or in combination is given in WO 00 69441, WO 02 36135, WO 03 039571 and PCT/GB2004/002319 which are incorporated by reference herein in their entirety.

There is still a need to provide further therapies mat allow an effective treatment of mammals, in particular humans, with ET-743 while reducing or eliminating its toxic side effects and minimizing further adverse effects.

### DRAWING OF THE INVENTION

FIG. 1 shows the Mean Plasma Concentration of ET-743 (also referred to as Trabectedin throughout the Examples) as a function of time after die start of the infusion; where FIG. 1A relates to results obtained from the present study and FIG. 1B relates to results presented in Van Kestern et al. (“Clinical Pharmacology of the novel marine-derived anticancer agent Ecteinascidin 743 administered as a 1- and 3-hour infusion in a phase I study; Anticancer Drugs; 13(4); 381-393; 2002”).

### SUMMARY OF THE INVENTION

This invention relates to combination products, combination drug treatments and methods for treating patients afflicted with cancer, having fewer and less severe unwanted toxic effects.

In accordance with one aspect, the invention provides a method of treating the human body for cancer comprising administering an effective therapeutic amount of a Pegylated Liposomal form of the anthracycline Doxorubicin (“PLD”), in combination with an effective therapeutic amount of ET-743. Preferably the mammal is a human.

### EMBODIMENTS OF THE INVENTION

Surprisingly, it has been found that the combination of ET743 and PLD can lead to increased anti-tumour efficacy while at the same time having reduced myelotoxicity and reduced cardiotoxicity. Furthermore, the combination of ET-743 with PLD is synergistic.

The PLD can be Doxorubicin hydrochloride (HCl) in Pegylated Liposomal form. The encapsulation in liposomes makes it suitable for intravenous administration. Liposomes are microscopic vesicles composed of a phospholipid bilayer that are capable of encapsulating active drugs. “Pegylation” is when liposomes are formulated with surface-bound methoxypolyethylene glycol (MPEG). Liposomal encapsulation may substantially affect a drug's functional properties relative to those of the unencapsulated drug. In addition, different liposomal drug products may vary from one another in the chemical composition and physical form of the liposomes. Such differences may substantially affect the functional properties of liposomal drug products. Doxil™ is an example of a commercially available form of Pegylated Liposomal Doxorubicin.

A combination of PLD and ET-743 is effective with reduced myelotoxicity and reduced cardiotoxicity in comparison with the toxicities observed using a combination of Doxorubicin and ET-743.

The increased anti-tumour efficacy is in comparison to treatments using ET743 alone. It has been found that the combination of PLD and ET-743 is tolerated to an extent in which both drugs may be administrated at full, or near full, therapeutic doses for prolonged periods of time.

In one aspect, the present invention is directed to a composition for the treatment of the human body for cancer, comprising ET-743 and PLD, which is effective with reduced toxicity in comparison with the toxicity observed using a combination of Doxorubicin and ET-743. In particular, the ET-743 and PLD combination shows reduced myelotoxicity and reduced cardiotoxicity.

In another aspect, the present invention is directed to a medical kit for administering ET-743 in combination with PLD, comprising a supply of ET-743 in dosage units for at least one cycle, wherein the dosage unit contains the appropriate amount of ET-743 for the treatments defined and a pharmaceutical acceptable carrier, and printed instructions for administering ET-743 according to a dosing schedule.

In another aspect, the invention is directed to the use of ET-743 in the preparation of a medicament for an effective treatment of the human body for cancer by combination therapy employing ET-743 with PLD.

The term “combination” as used throughout the specification, is meant to encompass the administration of the therapeutic agents in the same or separate pharmaceutical formulations, and at the same time or at different times.

In a further aspect, the invention is directed to die use of PLD in the preparation of a medicament for an effective treatment of the human body for cancer by combination therapy employing PLD with ET-743. The treatment is effective with reduced myelotoxicity and cardiotoxicity and is also notable for the absence of both mucositis and alopecia.

In a further aspect, the present invention is directed to a method for increasing anti-tumour efficacy of ET-743 in a treatment of the human body for cancer comprising administering an effective therapeutic amount of ET-743 in combination with an effective therapeutic amount of PLD.

The invention also provides a method of treating the human body for cancer comprising administering an effective therapeutic amount of PLD in combination with an effective therapeutic amount of ET-743. Preferably the mammal is a human.

The term “ET-743” is intended here to cover any pharmaceutically acceptable salt, ester, solvate, hydrate or any other compound which, upon administration to the recipient is capable of providing (directly or indirectly) the compound as described herein. However, it will be appreciated that non-pharmaceutically acceptable salts also fall within the scope of the invention since these may be useful in the preparation of pharmaceutically acceptable salts. The preparation of salts and prodrugs and derivatives can be carried out by methods known in the art.

ET-743 is supplied and stored as a sterile lyophilized product, consisting of ET-743 and excipient in a formulation adequate for therapeutic use, in particular a formulation containing mannitol and a phosphate salt buffered to an adequate pH.

Administration of the compositions of this method is suitably by intravenous injection. Administration can be carried out continuously or periodically within the maximum tolerated dose (MTD). Throughout the specification, MTD is intended to relate to the highest dose at which less with in one third of the subjects in a dose-level cohort experienced dose limiting toxicity (DLT).

ET-743 and PLD may be provided as separate medicaments for administration at the same time or at different times. Preferably. ET-743 and PLD are provided as separate medicaments for administration at different times. When administered separately and at different times, either ET-743 or PLD may be administered first; however, it is preferable to administer PLD followed by ET-743.

Typical infusion times are up to 72 hours, more preferably 1-24 hours, with 1-6 hours most preferred. When PLD and ET-743 are provided as separate medicaments for administration at different times, the infusion times for each may differ.

Infusion times for PLD are generally up to 6 hours, more preferably 1-3 hours, with 1-2 hours most preferred. Infusion times for ET-743 are generally up to 24 hours, more preferably about 1, about 3 or about 24 hours. Short infusion times which allow treatment to be carried out without an overnight stay in hospital are especially desirable.

It will be appreciated that the correct dosage of the compositions of this aspect of the invention will vary according to the particular formulation, the mode of application, and the particular situs, host and tumour being treated. Other factors like age, body weight, sex, diet, time of administration, rate of excretion, condition of the host, drug combinations, reaction sensitivities and severity of die disease shall be taken into account. All dosages Eire expressed in milligrams (mg) per metre square (m²) of body surface area. Since in this method of the invention PLD and ET-743 are used in combination, the dosage of each is adjusted to provide the optimum clinical response.

In the present method of the invention, dosages of PLD of up to 50 mg/m² are used, more preferably 25-45 mg/m², with 30-40 mg/m² most preferred, with about 30 mg/m² even most preferred. Dosages of ET-743 of up to 1.3 mg/m² are used, more preferably 0.4-1.2 mg/m², with about 1.1 mg/m² most preferred.

According to a preferred embodiment of this aspect of the invention, 25-45 mg/m² of PLD are administered intravenously followed by up to 1.3 mg ET-743, also administered intravenously. More preferably, about 30 mg/m² of PLD are administered followed by about 1.1 mg/m² ET-743. The PLD is preferably administered over an infusion time of up to 6 hours, more preferably 1-2 hours, most preferably 1 hour. The ET-743 is preferably administered over an infusion time of about 1, about 3 or about 24 hours.

The administration of the combination is performed in cycles in the application method of the invention. Intravenous infusions of PLD and ET-743 are given to the patients typically every 3 weeks, allowing for a resting phase in each cycle in which the patients recover. The preferred duration of each cycle is typically of 3 to 4 weeks; multiple cycles can be given as needed. Dose delays and/or dose reductions and schedule adjustments are performed as needed depending on individual patient tolerance of treatments.

According to a particularly preferred embodiment, every 3 weeks, about 30 mg/m² of PLD are administered to a patient over an infusion time of about 1 hour followed by administration of about 1.1 mg/m² of ET-743 over an infusion time of about 3 hours

By using a dosing regimen in accordance with that used in these preferred embodiments, it has been found that the combination is well tolerated when both drugs are administrated at full, or near full, therapeutic doses for prolonged periods of lime.

The full dose of ET-743 is known to be 1.3 mg/m² when administered as a single agent over 3 hours. The full dose of PLD as it is currently used in clinical practice is 10 mg/m² per week when administered as a single agent.

The following figures indicate that the use of PLD in combination with ET-743, allows an escalated dose of ET-743 to be tolerated, in comparison to when Doxorubicin is used. A phase I dose escalation study of Doxorubicin (60 mg/m²) in combination with ET-743, administered over 3 hours, could only support a ET-743 dose of 0.7 mg/m². In a Phase I dose escalation study of 30 mg/m² PLD in combination with ET-743 administered over 3 hours, ET-743 could be escalated to 1.1 mg/m².

Accordingly, in another aspect, the present invention is directed to a method for maximising the tolerated dose of ET-743 in a treatment of the human body for cancer comprising administering an effective therapeutic amount of ET-743 in combination with a Pegylated Liposomal form of Doxorubicin.

In summary, the combination of ET-743 and PLD allows for an effective cancer therapy in humans, with reduced myelotoxicity and cardiotoxicity. In phase I trials using ET-743 together with PLD, measurable responses demonstrated evidence of clinical benefit to patients with soft tissue sarcoma, and ovarian and head and neck cancer.

The markedly reduced cardiotoxicity exhibited means that the combinations for use in this aspect of the invention can be administered on a longterm basis. Furthermore, the combinations are notable for the absence of both mucositis and alopecia.

Example 1 shows the results of a study to evaluate the MTD of ET-743 in combination with 30 mg/m² of PLD, together with results of phase I trials. The MTD of ET743 in combination with 30 mg/m² of PLD was established as 1.1 mg/m² in the course of treatments.

In summary, this invention therefore provides methods of treatment, the use of the compounds in the preparation of a composition for treatment of cancer and related embodiments. The present invention also extends to the compositions of the invention for use in a method of treatment.

The present invention also relates to pharmaceutical preparations including a pharmaceutically acceptable carrier, which contain as active ingredient a compound or compounds of the invention, as well as the processes for their preparation.

The following example further illustrates the invention. It should not be interpreted as a limitation of the scope of the invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### EXAMPLES OF THE INVENTION

Throughout tire Example Ecteinascidin 743 (ET-743) is also referred to as Trabectedin.

### Example 1

A phase I trial combining PLD and trabectedin was performed. The objective of this study was to determine the maximum tolerated dose (MTD) of trabectidin in combination with PLD 30 mg/m² administered every 21 clays. Additional objectives were to evaluate the safety profile of this combination of drugs and to evaluate the pharmacokinetics of trabectidin and PDL when given in combination. The maximum tolerated dose (MTD) relates to the highest dose at which less than one third of the subjects in a dose-level cohort experienced dose-limiting toxicity (DLT).

We designed a dose finding trial with a fixed PLD dose of 30 mg/m² administered intravenously over one hour, followed immediately by one of six trabectedin doses (0.4, 0.6, 0.75, 0.9, 1.1, and 1.3 mg/m²) administered intravenously over 3 hours. This treatment was repeated every 21 days.

Entry-criteria included normal liver function tests, limited prior, doxorubicin-exposure (dose less than 250 mg/m²), normal cardiac function and Eastern Cooperative Oncology Group (ECOG) Performance Status (PS) score of 0 or 1.

Thirty patients, 14 with sarcomas, 2 with ovarian cancer, 2 with pancreatic cancer, 2 with head and neck carcinoma, 1 with bladder cancer, 1 with breast cancer, 1 with gastric cancer, 1 with NSCLC, 1 with SCLC and 5 more with other types of cancers have been treated (Table 1).

TABLE 1
Baseline Disease Characteristics
Time to 1st Dose Since Diagnosis, Mo

| | |
|---|---|
| N | 30 |
| Mean (SD) | 37.4 (43.8) |
| Median | 25.5 |
| Range | 1.2-216.0 |

Time to 1st Dose Since Last Chemo, Mo

| | |
|---|---|
| N | 22 |
| Mean (SD) | 3.54 (3.0) |
| Median | 2.6 |
| Range | 0.9-12.8 |

Histology

| | |
|---|---|
| N | 30 |
| Bladder | 1 |
| Breast | 1 |
| Head and Neck | 2 |
| NSCLC | 1 |
| Ovary | 2 |
| Pancreatic | 2 |
| Sarcoma | 14 |
| Gastric | 1 |
| SCLC | 1 |
| Other | 5 |

Nine patients of 30 had a Performance Status (PS) of 0 (Fully active, able to carry on all pre-disease performance without restriction). Table 2 shows demographic data of the patients treated.

TABLE 2
Demographic Data

Total

Sex, n (%)

| | |
|---|---|
| N | 30 |
| Female | 17 (57) |
| Male | 13 (43) |

Race, n (%)

| | |
|---|---|
| N | 30 |
| Black | 1 (3) |
| White | 29 (97) |

Age in Years

| | |
|---|---|
| N | 30 |

Category, n (%)

| | |
|---|---|
| 18-60 | 25 (83) |
| >60 | 5 (17) |
| Mean (SD) | 51.5 (13.8) |
| Median | 53.0 |
| Range | 20-78 |

Baseline ECOG Score, n (%)

| | |
|---|---|
| N | 30 |
| 0 | 9 (30) |
| 1 | 21 (70) |

Patients were heavily pretreated: 23/30 had received 1-5 prior chemotherapies (median 3), 15/30 prior radiotherapy, and 27/30 prior surgical resection (Table 3).

TABLE 3
Previous Therapy

N = 30

Previous Systemic Therapy, n (%)

| | |
|---|---|
| No | 7 (23) |
| Yes | 23 (77) |

Previous Surgery, n (%)

| | |
|---|---|
| No | 3 (10) |
| Yes | 27 (90) |

Previous Radiotherapy, n (%)

| | |
|---|---|
| No | 15 (50) |
| Yes | 15 (50) |

Tables 4a and 4b show the number (N) of patients exposed in each ET-743 dose, the treatment duration and the dose intensity.

TABLE 4a
Exposure to Treatment: Treatment Duration and Dose Intensity

| | | |
|---|---|---|
| ET743 | ET743 | ET743 |
| 0.4 mg/m² | 0.6 mg/m² | 0.75 mg/m² |
| (N = 3) | (N = 3) | (N = 3) |

Total Treatment Duration, Weeks

| | | | |
|---|---|---|---|
| Mean (SD) | 39.4 (37.4) | 31.5 (37.1) | 24.2 (31.7) |
| Median | 24.0 | 14.4 | 6.0 |
| Range | 12.1-82.0 | 6.0-74.0 | 5.9-60.9 |

Total Number of Cycles

| | | | |
|---|---|---|---|
| Mean (SD) | 13.0 (12.3) | 10.0 (12.2) | 7.7 (9.8) |
| Median | 8.0 | 4.0 | 2.0 |
| Range | 4-27 | 2-24 | 2-19 |

Overall Relative Dose Intensity ET-743

| | | | |
|---|---|---|---|
| Mean (SD) | 1.1 (0.2) | 0.9 (0.1) | 1.0 (0.0) |
| Median | 1.0 | 1.0 | 1.0 |
| Range | 1.0-1.3 | 0.8-1.0 | 0.9-1.0 |

Overall Relative Dose Intensity PLD

| | | | |
|---|---|---|---|
| Mean (SD) | 0.9 (0.1) | 0.9 (0.1) | 0.9 (0.2) |
| Median | 1.0 | 1.0 | 1.0 |
| Range | 0.8-1.0 | 0.8-1.0 | 0.7-1.0 |

TABLE 4b
Exposure to Treatment: Treatment Duration and Dose Intensity

| | | | |
|---|---|---|---|
| ET743 | ET743 | ET743 | |
| 0.9 mg/m² | 1.1 mg/m² | 1.3 mg/m² | Total |
| (N = 3) | (N = 12) | (N = 6) | (N = 30) |

Total Treatment Duration, Weeks

| | | | | |
|---|---|---|---|---|
| Mean (SD) | 6.0 (0.0) | 14.6 (11.1) | 25.7 (11.3) | 21.1 (20.5) |
| Median | 6.0 | 11.1 | 25.5 | 13.0 |
| Range | 6.0-6.0 | 3.0-42.0 | 7.1-39.9 | 3.0-82.0 |

Total Number of Cycles

| | | | | |
|---|---|---|---|---|
| Mean (SD) | 2.0 (0.0) | 4.8 (3.8) | 8.2 (3.8) | 6.8 (6.7) |
| Median | 2.0 | 3.5 | 8.0 | 4.0 |
| Range | 2-2 | 1-14 | 2-13 | 1-27 |

Overall Relative Dose Intensity ET-743

| | | | | |
|---|---|---|---|---|
| Mean (SD) | 1.1 (0.0) | 0.9 (0.1) | 0.8 (0.1) | 0.9 (0.1) |
| Median | 1.0 | 0.9 | 0.7 | 1.0 |
| Range | 0.9-1.0 | 0.8-1.0 | 0.7-1.0 | 0.7-1.3 |

Overall Relative Dose Intensity PLD

| | | | | |
|---|---|---|---|---|
| Mean (SD) | 1.0 (0.0) | 0.9 (0.1) | 0.8 (0.1) | 0.9 (0.1) |
| Median | 1.0 | 0.9 | 0.7 | 0.9 |
| Range | 0.9-1.0 | 0.7-1.0 | 0.7-0.9 | 0.7-1.0 |

Worst on-treatment drug related grade 3 and 4 toxicities have been minimal, limited to neutropenia and transaminases elevation. Tables 5a and 5b show the frequently reported drug-related Grade ¾ adverse events in least 5% of subjects. The adverse events reported at any time from first treatment dose up to 30 days after the last treatment dose are included. In order to define the toxicity grade, NCI common criteria are used.

TABLE 5a
Drug Related Adverse Events

| | ET743 | ET743 | ET743 |
|---|---|---|---|
| | 0.4 mg/m² | 0.6 mg/m² | 0.75 mg/m² |
| | (N = 3) | (N = 3) | (N = 3) |
| Total no, subjects with | 0 | 1 | 0 |
| Grade 3/4 drug- | | | |
| related AE | | | |
| Liver and Biliary | 0 | 0 | 0 |
| System | | | |
| SGPT Increased | 0 | 0 | 0 |
| SGOT Increased | 0 | 0 | 0 |
| Other | 0 | 1 | 0 |
| Palmar-Plantar | 0 | 0 | 0 |
| Erythrodysesthesia | | | |
| Allergic Reaction | 0 | 0 | 0 |
| Nausea | 0 | 1 | 0 |

TABLE 5b
Drug Related Adverse Events

| | ET743 | ET743 | ET743 | |
|---|---|---|---|---|
| | 0.9 mg/m² | 1.1 mg/m² | 1.3 mg/m² | Total |
| | (N = 3) | (N = 12) | (N = 6) | (N = 30) |
| Total no. subjects with | 0 | 10 | 6 | 17 |
| Grade 3/4 drug- | | | | |
| related AE | | | | |
| Liver and Biliary | 0 | 6 | 3 | 9 |
| System | | | | |
| SGPT Increased | 0 | 6 | 3* | 9 |
| SGOT Increased | 0 | 1 | 2* | 3 |
| Other | 0 | 4 | 2 | 7 |
| Palmar-Plantar | 0 | 2 | 2 | 4 |
| Erythrodysesthesia | | | | |
| Allergic Reaction | 0 | 2 | 0 | 2 |
| Nausea | 0 | 1 | 0 | 2 |

*DLT (dose-limiting toxicity): 2 patients experienced grade 4 elevation of SGPT during cycle 1.

In addition, Tables 6a and 6b show the drug-related serious adverse events reported.

TABLE 6a
Drug Related Serious Adverse Events

| | ET743 | ET743 | ET743 |
|---|---|---|---|
| | 0.4 mg/m² | 0.6 mg/m² | 0.75 mg/m² |
| | (N = 3) | (N = 3) | (N = 3) |
| Total no. subjects | 0 | 0 | 0 |
| with SAE | | | |
| Nausea/Vomiting | 0 | 0 | 0 |

TABLE 6b
Drug Related Serious Adverse Events

| | ET743 | ET743 | ET743 | |
|---|---|---|---|---|
| | 0.9 mg/m² | 1.1 mg/m² | 1.3 mg/m² | Total |
| | (N = 3) | (N = 12) | (N = 6) | (N = 30) |
| Total no. subjects | 0 | 1 | 0 | 1 |
| with SAE | | | | |
| Nausea/Vomiting | 0 | 1 | 0 | I |

Out of 18 subjects that discontinued treatment only one subject terminated treatment due to drug related adverse event (Tables 7a and 7b).

TABLE 7a
Subject Disposition/Reasons For Treatment Termination

| | ET743 | ET743 | ET743 |
|---|---|---|---|
| | 0.4 mg/m² | 0.6 mg/m² | 0.75 mg/m² |
| | (N = 3) | (N = 3) | (N = 3) |
| Ongoing (Cycles) | 1 (27+) | 1 (24+) | 1 (19+) |
| Terminated | 2 | 2 | 2 |
| Death | 0 | 0 | 0 |
| Drug-Related | 0 | 0 | 0 |
| AE (hand- | | | |
| foot syndrome) | | | |
| Disease | 2 | 1 | 2 |
| Progression | | | |
| Subject Choice | 0 | 1 | 0 |

TABLE 7b
Subject Disposition/Reasons For Treatment Termination

| | ET743 | ET743 | ET743 | |
|---|---|---|---|---|
| | 0.9 mg/m² | 1.1 mg/m² | 1.3 mg/m² | Total |
| | (N = 3) | (N = 12) | (N = 6) | (N = 30) |
| Ongoing (Cycles) | 0 | 6 (8+) | 3 (13+) | 12 |
| Terminated | 3 | 6 | 3 | 18 |
| Death | 0 | 0 | 1 | 1 |
| Drug-Related | 0 | 1 | 0 | 1 |
| AE (hand- | | | | |
| foot syndrome) | | | | |
| Disease | 3 | 5 | 2 | 15 |
| Progression | | | | |
| Subject Choice | 0 | 0 | 0 | 1 |

Five patients, three with soft tissue sarcoma, and one each of ovarian and head and neck cancer, had a partial response. Fourteen (14) additional patients (five with sarcoma, and one each carcinoid tumor, pancreatic, bladder, head and neck, thyroid, breast, gastric, SCLC and ovarian cancer) have had stable disease for > 3 months (Table 8a and 8b).

TABLE 8a
Best Overall Response

| | ET743 | ET743 | ET743 |
|---|---|---|---|
| Best | 0.4 mg/m² | 0.6 mg/m² | 0.75 mg/m² |
| Response | (N = 3) | (N = 3) | (N = 3) |
| PR | 1 Sarcoma | 0 | 0 |
| SD | 1 Pancreatic | 1 Sarcoma | 1 Thyroid |
| | 1 Carcinoid tumor | 1 Bladder | |
| | | 1 Head &Neck | |

TABLE 8b
Best Overall Response

| | ET743 | ET743 | ET743 |
|---|---|---|---|
| Best | 0.9 mg/m² | 1.1 mg/m² | 1.3 mg/m² |
| Response | (N = 3) | (N = 12) | (N = 6) |
| PR | 0 | 1 Sarcoma | 1 Papillary serous AC |
| | | (PNET) | 1 Sarcoma |
| | | | 1 Head &Neck |
| SD | 0 | 3 Sarcoma | 1 Ovarian |
| | | 1 Breast | 1 Sarcoma |
| | | 1 Gastric | |
| | | 1 SCLC | |

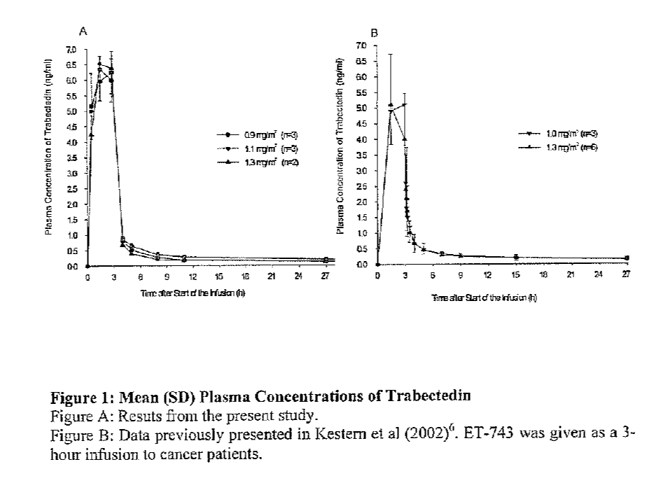

The concomitant administration of PLD does not have an impact on the pharmacokinetics (PK) of trabectedin. Based on preliminary pharmacokinetic analysis, the values of trabectedin CL (systematic clearance after i.v. dose), t½ (half life) and Vss (apparent volume of distribution at steady state) are within the range observed when trabectedin is given alone (historical control data) (FIG. 1, Table 9),

TABLE 9
Mean Noncompartmental Pharmacokinetic
Parameters of Trabectedin

| | Dose | | Cₘₐₓ | AUC_{∞} | Cl | Vss | T_{1/2} |
|---|---|---|---|---|---|---|---|
| | (mg/m²) | N | (ng/ml) | (ng*h/ml) | (l/h/m2) | (1/m²) | (h) |
| PLD 30 | 0.9 | 3 | 6 | 39 | 23 | 1425 | 93 |
| mg/m² | | | | | | | |
| 1 hour | 1.1 | 3 | 6 | 42 | 26 | 1113 | 65 |
| infusion + | | | | | | | |
| ET-743 | 1.3 | 2 | 7 | 31 | 44 | 962 | 47 |
| 3 hours | | | | | | | |
| infusion | | | | | | | |

From this study we conclude that the MTD of trabectidin is 1.1 mg/m² when is administered in combination with PLD 30 mg/m². It has been demonstrated that this combination is well tolerated when both drugs are administered at full (or near full) therapeutic doses for prolonged periods of time. The recommended dose of this combination treatment is 1.1 mg/m² of trabectidin plus 30 mg/m² of PDL.

In addition, it has been shown that the pharmacokinetics of trabectidin were overtly not impacted by concomitant administration of PDL.

1. A method of treating the human body for cancer comprising administering an effective therapeutic amount of a Pegylated Liposomal form of the anthracycline Doxorubicin (“PLD”), in combination with an effective therapeutic amount of ET-743.

2. The method according to claim 1, wherein said effective therapeutic amounts of ET-743 and PLD are administered as part of the same medicament or are provided as separate medicaments for administration at the same time or at different times.

3. The method according to claim 1 or claim 2, wherein said effective therapeutic amounts of ET-743 and PLD are provided as separate medicaments for administration at different times.

4. The method according to claim 3, wherein said effective therapeutic amount of PLD is administered prior to the administration of said effective therapeutic amount of ET-743.

5. The method according to any one of claims 1 to 4, wherein said effective therapeutic amounts of PLD and ET-743 are administered by intravenous injection.

6. The method according to claim 5, wherein the infusion time for intravenous injection is up to 6 hours for said effective therapeutic amount of PLD and up to 24 hours for said effective therapeutic amount of ET-743.

7. The method according to claim 6, wherein the infusion time for intravenous injection is 1-2 hours for said effective therapeutic amount of PLD and about 3 hours for said effective therapeutic amount of ET-743.

8. The method according to any one of claims 5 to 7, where the infusions are carried out at an interval of 3 to 4 weeks.

9. The method according to any one of the preceding claims, wherein an effective therapeutic amount of PLD is administered in a dosage of up to 50 mg/m², followed by an effective therapeutic amount of ET-743 administered in a dosage of up to 1.3 mg/m².

10. The method according to claim 9, wherein said effective therapeutic amount of PLD is administered in a dosage of 30-40 mg/m² over an infusion time of 1-2 hours followed by said effective therapeutic amount of ET-743 administered in a dosage of about 1.1 mg/m² over an infusion time of about 3 hours.

11. The method according to claim 10, wherein said effective therapeutic amount of PLD is administered in a dosage of about 30 mg/m² over an infusion time of about 1 hour followed by said effective therapeutic amount of ET-743 administered in a dosage of about 1.1 mg/m² over an infusion time of about 3 hours.

12. The use of ET-743 in the preparation of a medicament for an effective treatment of the human body for cancer by combination therapy employing ET-743 with a Pegylated Liposomal form of the anthracycline Doxorubicin (“PLD”).

13. The use of PLD in the preparation of a medicament for an effective treatment of the human body for cancer by combination therapy employing PLD with ET-743.

14. The use according to claim 12 or claim 13, wherein the combination of ET-743 with PLD is synergistic.

15. The use according to any of claims 12 to 14, wherein the ET-743 forms part of the same medicament, or is provided as a separate medicament for administration at the same time or a different time as PLD.

16. The use according to any of claims 12 to 15, wherein the patient has a cancer selected from soft tissue sarcoma, ovarian cancer and head and neck cancer.

17. A composition for the treatment of the human body for cancer, comprising ET-743 and PLD, which is effective with reduced toxicity in comparison with the toxicity observed using a combination of ET-743 and Doxorubicin.

18. A medical kit for administering ET-743 in combination with PLD, comprising a supply of ET-743 in dosage units for at least one cycle, wherein the dosage unit contains the appropriate amount of ET-743 for the treatments defined and a pharmaceutically acceptable carrier, and printed instructions for administering ET-743 according to a dosing schedule.

19. A method for increasing anti-tumour efficacy of ET-743 in a treatment of the human body for cancer comprising administering an effective therapeutic amount of ET-743 in combination with an effective therapeutic amount of PLD.

20. A method for maximising the tolerated dose of ET-743 in a treatment of the human body for cancer comprising administering an effective therapeutic amount of ET-743 in combination with a Pegylated Liposomal form of the anthracycline Doxorubicin (“PLD”).

21. A medical kit for administering ET-743 in combination with PLD, comprising a supply of ET-743 in dosage units, wherein each of said dosage units contains an amount of ET-743 for its therapeutically effective administration in combination with PLD.

22. A medical kit for administering PLD in combination with ET-743, comprising a supply of PLD in dosage units, wherein each of said dosage units contains an amount of PLD for its therapeutically effective administration in combination with ET-743.

23. A medical kit for administering ET-743 in combination with PLD, comprising both a supply of ET-743 according to claim 21 and a supply of PLD according to claim 22.

24. A medical kit according to claim 21 or 23, further comprising instructions for administering ET-743 according to a dosing schedule.

25. A medical kit according to claim 22 or 23, further comprising instructions for administering PLD according to a dosing schedule.

26. A combination for the treatment of the human body for cancer, comprising an effective therapeutic amounts of ET-743 and PLD which are part of the same medicament or are provided as separate medicaments for the administration at the same time or at different times.

27. A combination according to claim 26, wherein said effective therapeutic amounts of ET-743 and PLD are provided as separate medicaments for the administration at tire same time or at different times.

28. A combination according to claim 27, wherein said effective therapeutic amounts of ET-743 and PLD are provided as separate medicaments for the administration at different times.

29. A combination according to claim 28, wherein said effective amount of PLD is administered prior to the administration of said effective therapeutic amount of ET-743.

30. A combination according to any one of claims 26 to 29, wherein said effective therapeutic amounts of PLD and ET-743 are administered by intravenous injection.
